(19) United States
(12) Patent Application Publication
(10) Pub. No.: US 2006/0128970 A1
(43) Pub. Date: Jun. 15, 2006

(54) 3-Pyrrolidin-2-yl-propionic acid derivatives

(76) Inventors: Fritz Bliss, Hartheim (DE); Michel Lalonde, Basel (CH); Rudolf Schmid, Basel (CH); Rene Trussardi, Birsfelden (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/301,958

(22) Filed: Dec. 13, 2005

(30) Foreign Application Priority Data
Dec. 13, 2004 (EP) 04106514.5

(51) Int. Cl. C07D 207/12

(52) U.S. Cl. 548/531

(57) ABSTRACT

The present invention relates to the manufacture of the compounds of formula (I) 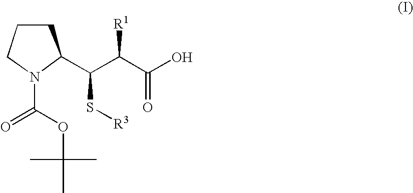 said compounds of formula (I) being valuable intermediates in the manufacture of Dolastatin 10 analogues, which are useful in the treatment of cancer.

### PRIORITY TO RELATED APPLICATIONS

This application claims the benefit of European Application No. 04106514.5, filed Dec. 13, 2004, which is hereby incorporated by reference in its entirety.

### FIELD OF THE INVENTION

The present invention relates to a new process for the manufacture of derivatives of 3-pyrrolidin-2-yl-propionic acid. According to the present invention, said derivatives are obtainable using two different reaction sequences A and B which require the same starting material.

### BACKGROUND OF THE INVENTION

The compounds obtainable by the process according to the present invention are valuable intermediates in the manufacture of Dolastatin 10 analogues. Dolastatin 10 is known to be a potent antimitotic peptide, isolated from the marine mollusk Dolabella auricularia, which inhibits tubulin polymerization and is a different chemical class from taxanes and vincas (Curr. Pharm. Des. 1999, 5: 139-162). Preclinical studies of Dolastatin 10 have demonstrated activities against a variety of murine and human tumors in cell cultures and animal models. Dolastatin 10 and two synthetic dolastatin derivatives, Cemadotin and TZT-1027 are described in Drugs of the future 1999, 24(4): 404-409.

Subsequently it had been found that certain Dolastatin 10 derivatives having various thio-groups at the dolaproine part show significantly improved anti-tumor activity and therapeutic index in human cancer xenograft models (WO 03/008378). However the synthesis disclosed in WO 03/008378 suffers from low yields, mainly due to laborious separation of the diastereoisomer mixtures, obtained in the β-addition reaction (s. scheme 1, below), by chromatography. Therefore, there remains a need to provide new and improved processes.

### SUMMARY OF THE INVENTION

The present invention addresses this problem by providing new, improved processes for the manufacture of compounds of the general formula (I), which are key fragments in the synthesis of the above-mentioned Dolastatin 10 derivatives. The compounds of general formula (I) made by the processes according to the present invention are useful as valuable intermediates in the manufacture of Dolastatin 10 analogues. It has now surprisingly been found that the process of the present invention provides an improved diastereoisomer ratio and an improved yield of the compounds of formula (I), which is subsequently retained in the synthesis of said Dolastatin 10 derivatives. Furthermore, the processes according to the present invention avoid the laborious separation of the diastereoisomer mixtures by chromatography. In particular the present invention relates to the manufacture of the compounds of formula (I) wherein formula I is: 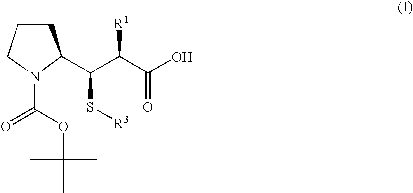
wherein R¹ and R³ represent alkyl.

In one embodiment (referred to herein as “sequence A”), the manufacture of the compounds of formula (I) comprises the following steps:
(a) reacting a compound of formula (II): 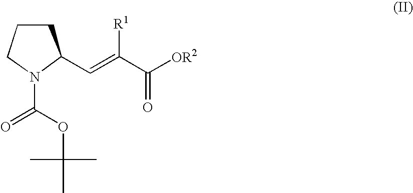
with a compound of formula (III):
   KS—R (III),
in the presence of triethylammonium chloride in a suitable solvent; wherein said compound of formula (III) can optionally be generated in situ by reacting a compound of formula (III-A) in the presence of potassium bases wherein formula (III-A) is: 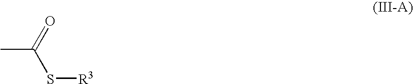
(b) chemically cleaving R² in the —COOR² ester group of the reaction product of step (a);
(c) adding an amine of formula NHR⁴R⁵ to the resulting carboxylic acid of step (b) to form an ammonium salt of formula (IV): 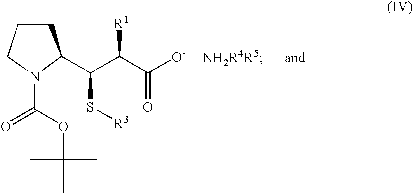
(d) decomposing said salt of formula (IV) in step (c) to form a compound of formula I;
wherein:
R¹, R³ and R⁶ independently from each other represent alkyl;
R² is benzyl or substituted benzyl; and
R⁴ and R⁵ are independently selected from cycloalkyl or alkyl, wherein said alkyl can be unsubstituted or substituted one, two or three times with hydroxy, alkoxy, amino, mono-alkylamino, di-alkylamino, acetoxy, alkylcarbonyloxy, carbamoyloxy, alkoxycarbonyl, carbamoyl, alkylcarbamoyloxy, halogen, cycloalkyl or phenyl.

Alternatively, in another embodiment (referred to herein as “sequence B”), the manufacture of the compounds of formula (I) comprises the following steps:
(a) reacting a compound of formula (V): 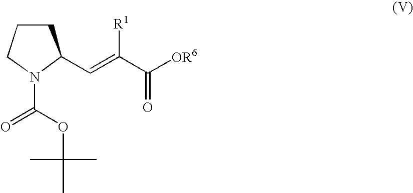
with a compound of formula (III):
   KS—R³ (III),
in the presence of triethylammonium chloride in a suitable solvent; wherein said compound of formula (III) can be optionally generated in situ by reacting a compound of formula (III-A) in the presence of potassium bases wherein formula III-A is: 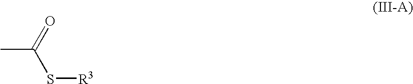
(b) adding hydrochloric acid to the reaction product of step (a) to form a compound of formula (VI): 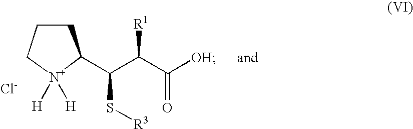
(c) reacting the reaction product of step (b) with a tert-butoxycarbonyl-delivering reagent to form a compound of formula I; wherein:
R¹, R³ and R⁶ independently from each other represent alkyl;
R² is benzyl or substituted benzyl; and
R⁴ and R⁵ are independently selected from cycloalkyl or alkyl, wherein said alkyl can be unsubstituted or substituted one, two or three times with hydroxy, alkoxy, amino, mono-alkylamino; di-alkylamino, acetoxy, alkylcarbonyloxy, carbamoyloxy, alkoxycarbonyl, carbamoyl, alkylcarbamoyloxy, halogen, cycloalkyl or phenyl.

The present invention also provides the compounds of formula (I) made by the manufacturing processes described above. In addition, the compounds of the general formulae (IV) and (VI) are new compounds and are further embodiments of the present invention.

### DETAILED DESCRIPTION OF THE INVENTION

As used herein, the term “alkyl” means a straight-chain or branched-chain hydrocarbon group containing a maximum of 8, preferably a maximum of 5, carbon atoms, e.g., methyl, ethyl, n-propyl, 2-methylpropyl (iso-butyl), 1-methylethyl (iso-propyl), n-butyl, 1,1-dimethylethyl (t-butyl or tert-butyl) or t-pentyl, and more preferably a maximum of 4 carbon atoms. The alkyl group may be unsubstituted or may be substituted with one or more substituents, preferably with one to three substituents, most preferably with one substituent. The substituents are selected from the group consisting of hydroxy, alkoxy, amino, mono-alkylamino, di-alkylamino, acetoxy, alkylcarbonyloxy, carbamoyloxy, alkoxycarbonyl, carbamoyl, alkylcarbamoyloxy, halogen, cycloalkyl and phenyl.

As used herein, the term “alkoxy” means —O-alkyl, wherein “alkyl” has the meaning given above.

As used herein, the term “acetoxy” refers to the group —O—C(O)—CH₃.

As used herein, the term “cycloalkyl” means a saturated mono- or bicyclic hydrocarbon group, containing from 3 to 10 carbon atoms, preferably from 3 to 7 carbon atoms, and more preferably 5 or 6 carbon atoms. Examples of such cycloalkyls are cyclopropyl, cyclopentyl, cyclohexyl, cycloheptyl and decahydro-naphthalene.

As used herein, the term “carbamoyl” refers to the group —CO—NH₂ and the term “carbamoyloxy” refers to the group —O—C(O)—NH.

As used herein, the term “alkylcarbamoyloxy” refers to an alkyl group as defined above attached to a parent structure via a carbymoyloxy radical, such as alkyl-NH—C(O)—O—.

As used herein, the term “alkylcarbonyloxy” refers to an alkyl group as defined above attached to a parent structure via a carbonyloxy radical, such as alkyl-C(O)—O—.

As used herein, in reference to R⁷, the term “phenyl(C₁-C₄)alkylamino” refers to a phenylalkylamino wherein the alkyl is a bivalent branched or unbranched hydrocarbon chain containing 1 to 4 carbon atoms such as methylene, ethylene, propylene, or butylene.

As used herein, in reference to R⁷, the term “phenyldi(C₁-C₄)alkylamino” refers to a phenyldialkylamino wherein one alkyl is a bivalent branched or unbranched hydrocarbon chain containing 1 to 4 carbon atoms such as methylene, ethylene, propylene, or butylene and the other is an alkyl group as defined herein before, containing 1 to 4 carbon atoms.

As used herein, in reference to R⁷, the term “phenyl(C₁-C₄)alkyloxy” refers to a phenylalkyloxy wherein the alkyl is a bivalent branched or unbranched hydrocarbon chain containing 1 to 4 carbon atoms such as methylene, ethylene, propylene, or butylene.

As used herein, the term “halogen” refers to fluorine, bromine, iodine and chlorine.

As used herein, the term “substituted benzyl” means a benzyl group, wherein the phenyl ring is one, two or three times substituted with a substituent independently selected from methyl, methoxy, phenyl, nitro, halogen or methylene-dioxy. Especially preferred are the following substitution patterns: 2,4,6-trimethyl, 3-methoxy, 4-methoxy, 2,4-dimethoxy, 3,4-dimethoxy, 3,5-dimethoxy, 2-nitro, 4-nitro, 2,4-dinitro, 4-bromo, 4-phenyl and 3,4-methylene-dioxy.

As used herein, the term “potassium bases” means basic potassium compounds, which are generally well known to the skilled artisan. Such potassium bases are for example potassium amides, potassium alkoxides or potassium hydroxide. Especially preferred according to the present invention is the use of potassium ethoxide.

As used herein, the term “tert-butoxycarbonyl-delivering reagent” means a reagent for the introduction of the N-Boc group as described below. Such “tert-butoxycarbonyl-delivering reagents” are well known to the skilled artisan and are, for example, described in “Protective Groups in Organic Synthesis,” 3^{rd}. Edition; Eds. T. W. Greene, P. G. M-Wuts, John Wiley & Sons, Inc., New York (1999); p. 518. A preferred “tert-butoxycarbonyl-delivering reagent” according to the present invention is di-tert-butyl dicarbonate.

As used herein, the terms “decompose,” “decomposing” and “decomposition,” and the like, refer to a chemical change. For example, the decomposition of the carboxylic acid ammonium salt of formula (IV) to obtain a compound of formula (I) may be performed by acidification with an inorganic aqueous acid such as hydrochloric or sulfuric acid and extraction of the compound of the formula (I) into an suitable organic solvent such as tert-butyl methyl ether, ethyl acetate or dichloromethane. Alternatively, the salt can be dissolved in an alkaline aqueous solution, for example, an aqueous solution of NaOH, KOH, Na₂CO₃, or K₂CO₃, and, after removal of the amine by extraction with a suitable organic solvent, the alkaline solution can be acidified with an inorganic acid such as hydrochloric or sulfuric acid and the compound of formula I extracted into a suitable organic solvent such as tert-butyl methyl ether, ethyl acetate or dichloromethane.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

As used herein, the term “suitable solvent” needs to be differentiated according to the different reaction sequences (sequence A and sequence B) as well as the different reaction steps within each sequence, according to scheme 1 below. In particular, the following solvents are “suitable” according to the various reaction steps of each sequence:

For Sequence A:

The β-addition is preferably carried out in ethers, such as tetrahydrofuran, methyl-tetrahydrofuran, tert-butyl methyl ether, dimethylether, diethylether and at temperatures from −20° C. to the reflux temperature of the respective solvent, most preferably between 0° C. to room temperature.

The ester cleavage is preferably carried out by hydrogenolysis in alcohols such as ethanol, methanol, isopropanol and the like; esters such as ethyl acetate, methyl acetate or isopropyl acetate; hydrocarbons such as toluene; or mixtures of the above solvents. This reaction requires temperatures between 0° C. to reflux temperature of the respective solvent, preferably from 0° C. to room temperature, whereby room temperature is most preferred.

The ammonium salt formation preferably takes place in solvents which provide suitable solubilities for compounds of formulae (I), NHR⁴R⁵ and (IV). In this connection ethers such as tert-butyl methyl ether, tetrahydrofuran, methyl-tetrahydrofuran, dimethylether, diethylether; alkanes such as hexane, cyclohexene, heptane; or aromatic solvents such as toluene, xylene; or mixtures of all the above-mentioned solvents, are especially preferred. The temperature can vary between −20° C. and 50° C., whereby the crystallization preferably occurs at temperatures between room temperature and −20° C.; most preferably at temperatures between 0° C. and −20° C.

The final decomposition of the isolated salt can take place under basic or acidic conditions. If basic conditions are used, inorganic bases such as alkali-hydroxides, -hydrogencarbonates or -carbonates are especially preferred. If acidic conditions are used, mineral acids such as hydrochloric acid, sulfuric acid are especially preferred. Said decomposition is carried out in any inert organic solvent immiscible with water, preferably in tert-butyl methyl ether, toluene or ethyl acetate and at temperatures between 0° C. and room temperature, most preferably at room temperature.

For Sequence B:

The solvents for the β-addition are as defined above for sequence A.

The reaction with hydrochloric acid takes place in solvents wherein the compounds of formula (VI) crystallize, preferably in esters, ethers or haloalkanes such as dichloromethane, more preferably in esters such as ethyl acetate; and at temperatures from 50° C. to −20° C., preferably from room temperature to −20° C. The crystallization preferably occurs at temperatures between 0° C. and −20° C.

The subsequent N-bocylation can be carried out with a tert-butoxycarbonyl-delivering reagent as defined above. A preferred method for the introduction of the N-Boc group involves the use of di-tert-butyl dicarbonate as reagent in the presence of a base, e.g. an inorganic base such as alkali metal hydroxide, -hydrogencarbonate, -carbonate; or tertiary amine bases such as trialkylamines, e.g. triethylamine. Suitable solvents for this reaction are polar solvents, especially water; alcohols; ethers such as tetrahydrofuran, dioxane and the like; haloalkanes such as dichloromethane; acetonitrile etc. The temperature can range from 0° C. to 50° C., whereby room temperature is especially preferred.

An embodiment of the present invention, is the process for the manufacture of the compounds of formula (I) comprising the following steps:
(a) reacting a compound of formula (II-A): 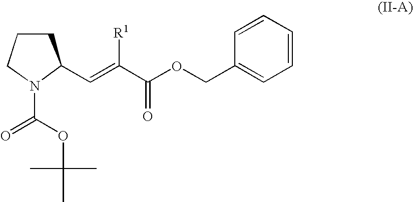
with: (1) a compound of formula (III) in the presence of triethylammonium chloride in tetrahydrofuran; or (2) a compound of formula (III-A) together with a potassium base as defined above, in the presence of triethylammonium chloride in tetrahydrofuran;
(b) chemically cleaving the benzyl-ester group from the reaction product of step (a);
(c) adding an amine of the formula NHR⁴R⁵ to the resulting carboxylic acid of step (b);
(d) adding a base to the reaction product of step (c); and
(e) adding one or more mineral acids to the reaction product of step (d);
wherein:
R¹ represents alkyl; and
R⁴ and R⁵ are independently selected from cycloalkyl or alkyl, wherein said alkyl can be unsubstituted or substituted one, two or three times with hydroxy, alkoxy, amino, mono-alkylamino, di-alkylamino, acetoxy, alkylcarbonyloxy, carbamoyloxy, alkoxycarbonyl, carbamoyl, alkylcarbamoyloxy, halogen, cycloalkyl or phenyl.

Another embodiment of the present invention is the process as described above, wherein the amines of formula NHR⁴R⁵ are selected from the group consisting of: dicyclohexylamine, diisopropylamine, (R)-α-phenylethylamine, benzyl-(R)-α-phenylethylamine and (R)-α-cyclohexylethylamine.

Still another embodiment of the present invention, is the process as described above, comprising the following steps:
(a) reacting the compound of formula (2): 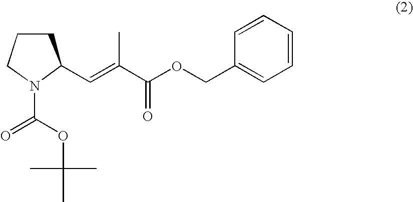
with S-methyl thioacetate together with potassium ethoxide, in the presence of triethylammonium chloride in tetrahydrofuran; and
(b) chemically cleaving the benzyl-ester group from the reaction product of step (a) to obtain the compound of formula (1a): 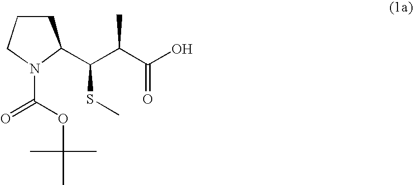
(c) adding dicyclohexylamine to the resulting carboxylic acid of step (b);
(d) adding sodium carbonate to the reaction product of step (c); and
(e) adding sulfuric acid to the reaction product of step (d).

Yet another embodiment of the present invention, is the process for the manufacture of the compounds of formula (I) comprising the following steps:
(a) reacting a compound of formula (V-A): 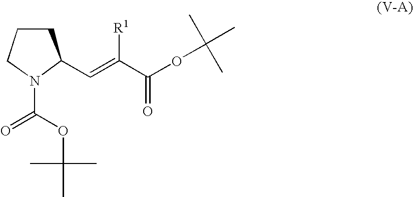
with: (1) a compound of formula (III) in the presence of triethylammonium chloride in tetrahydrofuran; or (2) a compound of formula (III-A) together with a potassium base as defined above, in the presence of triethylammonium chloride in tetrahydrofuran;
(b) reacting the reaction product of step (a) with dry hydrochloric acid in ethyl acetate,
(c) adding sodium carbonate to the reaction product of step (b); and
(d) reacting the reaction product of step (c) with di-tert-butyl dicarbonate;
wherein R¹ represents alkyl.

Still another embodiment of the present invention is the process as described above, comprising:
(a) reacting a compound of formula (4): 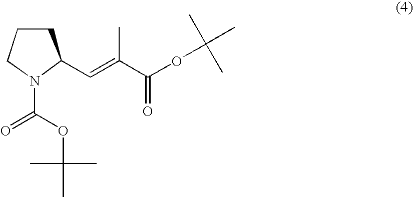
with S-methyl thioacetate together with potassium ethoxide, in the presence of triethylammonium chloride in tetrahydrofuran;
(b) reacting the reaction product of step (a) with dry hydrochloric acid in ethyl acetate;
(c) adding sodium carbonate to the reaction product of step (b); and
(d) reacting the reaction product of step (c) with di-tert-butyl dicarbonate to obtain the compound of formula (1a):

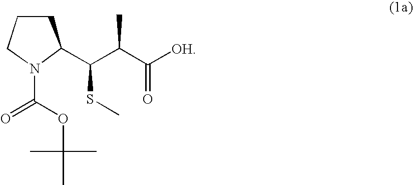

Still another embodiment of the present invention is the process as described above, wherein the compounds of formula (I) are further reacted to give the compounds of formula (A):

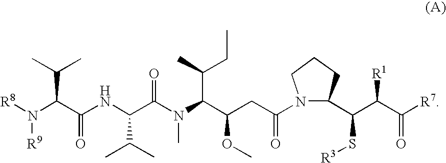

The manufacture of the compounds of formula (A) may comprise the following steps:
(a) reacting the compounds of formula (I) with an alcohol or an amine, and then chemically cleaving the tert-butoxycarbonyl group at the pyrrolidine N-atom, to give the compounds of formula (B): 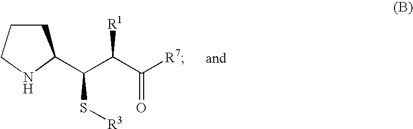
(b) further reacting the compounds of formula (B) with the compounds of formula (C): 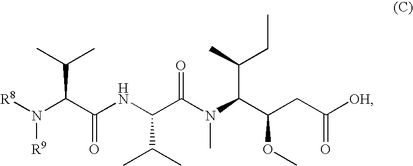
to give the compounds of formula (A); and wherein:
R¹ and R³ independently from each other represent alkyl;
R⁸ and R⁹ independently from each other represent alkyl; and
R⁷ is phenyl(C₁-C₄)alkyl-, or phenyldi(C₁-C₄)alkylamino or phenyl(C₁-C₄)alkyloxy, wherein the phenyl group optionally may be substituted with one, two or three substituents selected from the group consisting of halogen, alkoxycarbonyl, sulfamoyl, alkylcarbonyloxy, carbamoyloxy, cyano, mono-alkylamino, di-alkylamino, alkyl, alkoxy, phenyl, phenoxy, trifluoromethyl, trifluoromethoxy, alkylthio, hydroxy, alkylcarbonylamino, 1,3-dioxolyl, 1,4-dioxolyl, amino and benzyl.

Still another embodiment of the present invention is the process as described above for the manufacture of the compound of formula (A-1):

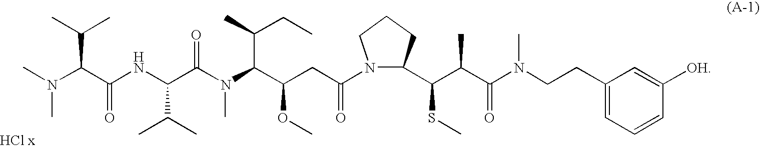

The manufacture of the compounds of formula (A-1) may comprise the following steps:
(a) reacting the compound of formula (1a): 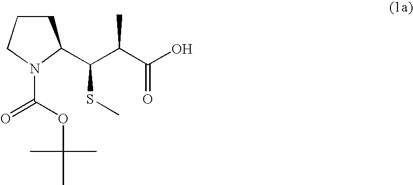
with 3-(2-methylamino-ethyl)-phenol;
(b) chemically cleaving the tert-butoxycarbonyl group at the pyrrolidine N-atom, to obtain the compound of formula (B-1): (B-1); and 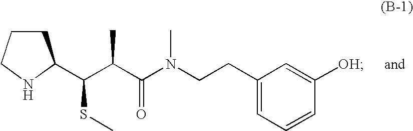
(c) reacting the compound of formula (B-1) with the compound of formula (C-1): 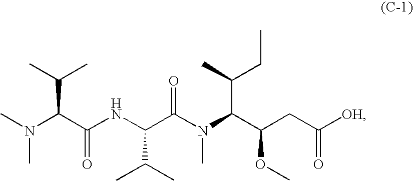
to obtain the compound of formula (A-1).
   Another embodiment of the present invention is a compound of formula (A) or a pharmaceutically acceptable salt thereof made by the process described above for the manufacture of compounds of formula (A). Another embodiment of the present invention is a compound of formula (A-1) made by the process described above for the manufacture of compounds of formula (A-1).

Compounds of formula (A) or (A-1) or their pharmaceutically acceptable salts made by the processes described above for the manufacture of compounds of formula (A) and (A-1) can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions. Such pharmaceutical compositions may be used for the inhibition of tumor growth or for the treatment of cancer.

The above-mentioned pharmaceutical compositions can be obtained by processing the compounds of formula (A) or (A-1) or their pharmaceutically acceptable salts made by the processes described above with pharmaceutically inert, inorganic or organic carriers. For example, lactose, corn starch or derivatives thereof, talc, stearic acids or it's salts and the like can The above-mentioned pharmaceutical compositions can be obtained by processing the be used as carriers for tablets, coated tablets, dragées and hard gelatine capsules. Suitable carriers for soft gelatine capsules are, for example, vegetable oils, waxes, fats, semi-solid and liquid polyols and the like. However, depending on the nature of the active substance, carriers may not be required for some soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They may also contain other therapeutically valuable substances.

In another embodiment of the present invention, there are provided the compounds of formula (IV): 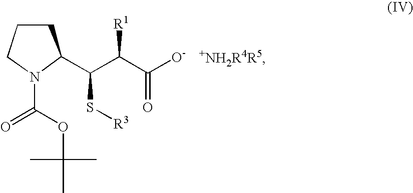
wherein:
R¹ and R³ independently from each other represent alkyl; and
R⁴ and R⁵ independently represent cycloalkyl or alkyl, wherein said alkyl can be unsubstituted, or substituted one, two or three times with hydroxy, alkoxy, amino, mono-alkylamino, di-alkylamino, acetoxy, alkylcarbonyloxy, carbamoyloxy, alkoxycarbonyl, carbamoyl, alkylcarbamoyloxy, halogen, cycloalkyl or phenyl.

In yet another embodiment of the present invention, there are provided the compounds of formula (IV), wherein R¹ and R³ are methyl; and the group ⁺NH₂R⁴R⁵ represents a cation selected from the group consisting of: dicyclohexylammonium, diisopropylammonium, (R)-α-phenylethylammonium, benzyl-(R)-α-phenylethylammonium, and (R)-α-cyclohexylethylammonium.

In still another embodiment of the present invention, there are provided the compounds of formula (VI): 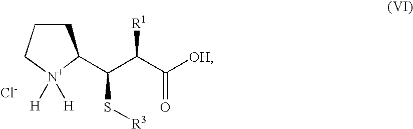
wherein:
R¹ and R³ independently from each other represent alkyl.

In still another embodiment of the present invention, there are provided the compounds as described above, wherein R¹ and R³ are methyl.

Yet another embodiment of the present invention is the compound (S)-2-((1R,2S)-2-Carboxy-1-methylsulfanyl-propyl)-pyrrolidine-1-carboxylic acid tert-butyl ester [formula (1a)].

A further embodiment of the present invention is the use of a compound of the formulae (IV), (VI) or (1a) as defined above in a process for the manufacture of formulae (I), (A) or (A-1).

Still another embodiment of the present invention is the use of a compound of the formulae (IV), (VI) or (1a) as defined above in the manufacture of the compounds of formula (A) as defined herein before.

Still another embodiment of the present invention is the use of a compound of formula (IV), wherein R¹ and R³ are methyl for the manufacture of formulae (I), (A) or (A-1); or the use of a compound of formula (1a) as defined above in the manufacture of the compound of formula (A-1) as defined herein before.

The process of the present invention can be performed according to the following general reaction scheme (scheme 1), wherein unless explicitly otherwise stated R¹, R³, R⁴ and R⁵ are defined in the same way as they are defined above in sequence A or sequence B for the manufacture of the compounds of formula (I). R¹⁰ is benzyl, substituted benzyl or alkyl, preferably benzyl or tert-butyl. 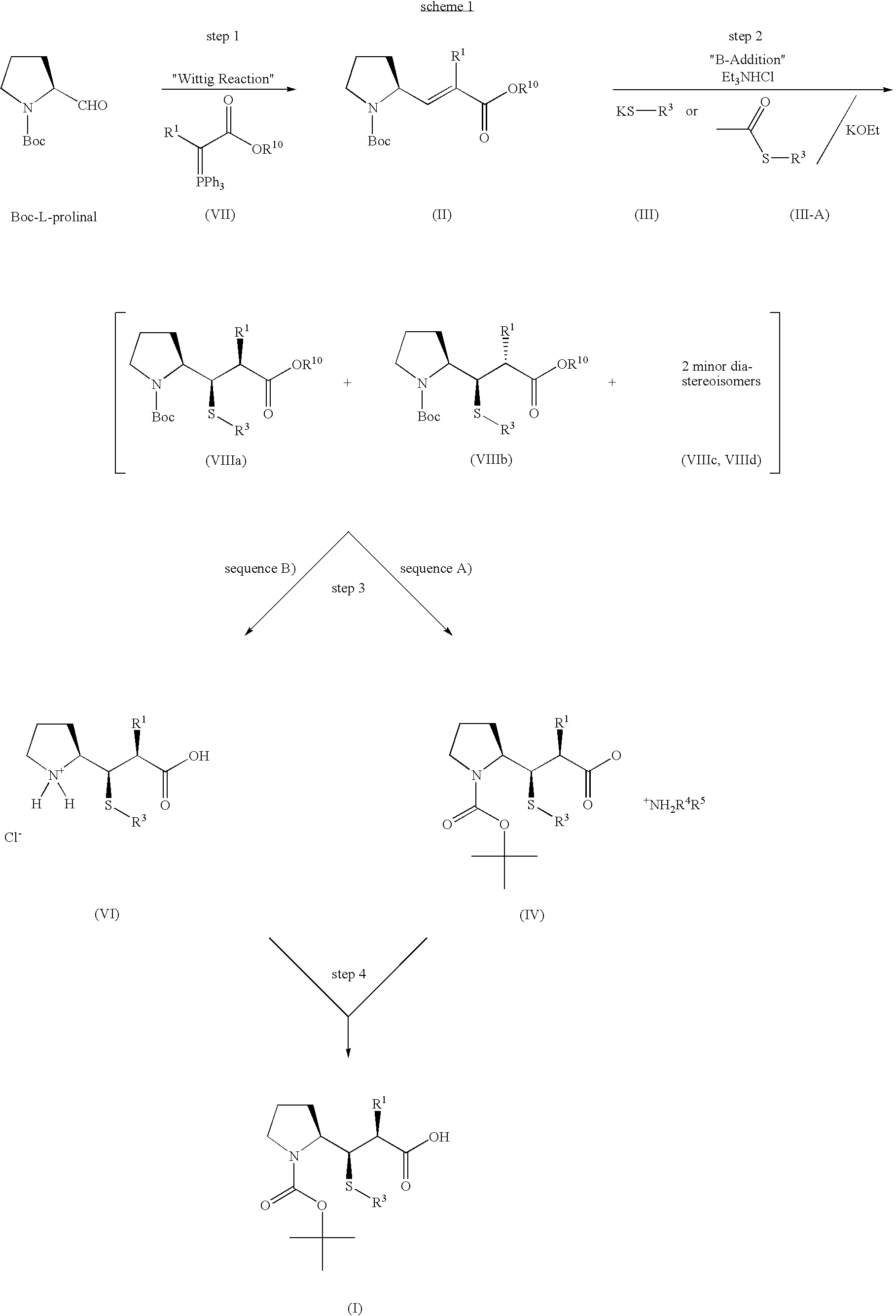
Step 1:

This step represents a Wittig reaction starting from commercially available tert-butoxycarbonyl protected L-prolinal (Boc-L-prolinal) with the ylide (VII) and using methods known to the skilled artisan (see e.g. Heterocycles, 36 (9), 1993, 2073-2080 and WO 03/008378). Said ylide wherein R¹ is methyl and R¹⁰ is benzyl can be obtained according to the synthesis described in “Y. Ito, M. Okano, R. Oda, Tetrahedron, 23, 1967, 2137.” Said ylide wherein R¹ is methyl and R¹⁰ is tert-butyl can be obtained according to the synthesis described in “Y. Guindon, L. Murtagh, V. Caron, S. R. Landry, G. Jung, M. Bencheqroun, A.-M. Faucher, B. Guerin, J. Org. Chem., 66, 2001, 5427” or “P. L. Stotter, K. A. Hill, Tetrahedron Lett., 16, 1975, 1679.”

Step 2:

This reaction is a β-addition of alkyl-mercaptanes, especially methyl mercaptane, wherein the potassium salts of formula (III) can be used as such, or generated in situ by adding the compounds of formula (III-A) in the presence of potassium bases, especially potassium ethoxide. According to the present invention, improvement of diastereoselectivity in this addition reaction is achieved by using triethylammonium chloride (Et₃N×HCl) as the proton source, compared to other common proton sources tested (see Table 1).

TABLE 1
Diastereoselectivity of β-Addition: Influence of Proton Source

| | AcSMe/KOEt/ | 3a/3b ^{a)} |
|---|---|---|
| Proton Source (X-H) | X-H equiv. | 2 h |
| phenol | 6/6/3 | 70:30 |
| succinimide | 6/6/3 | 83:17 |
| N-hydroxysuccinimide | 6/6/3 | 85:15 |
| Et₃N × HCl | 6/6/3 | 85:15 |
| Et₃N × HCl | 3/3/1.5 | 90:10 |
| CH₃SH | 6/3 ^{b)} | 88:12 |
| Et₃N × HCl | 3/1.5/1.0 ^{c)} | 89:11 |

^{a)} Ratio determined by GC analysis.
^{b)} Methyl mercaptan (6 equiv.) used instead of S-methyl thioacetate (III-A, scheme 1), no additional proton source.
^{c)} Methyl mercaptan (3 equiv.) used instead of S-methyl thioacetate (III-A, scheme 1); Et₃N × HCl (1.0 equiv.) as additional proton source.

Step 3:

With respect to reaction sequence A, the crude ester (mixture of VIII a, b, c and d, scheme 1 with R¹⁰ being benzyl or substituted benzyl) is now hydrogenolyzed, preferably in the presence of 20% Pd—C (30% w/w) in ethanol. According to the present invention, further treatment with the amines mentioned herein before, especially dicyclohexylamine in tert-butyl methyl ether, furnish the respective ammonium salts in good diastereoisomeric purities and high yields.

With respect to reaction sequence B the crude ester (mixture of VIII a, b, c and d, scheme 1 with R¹⁰ being alkyl, preferably tert-butyl) can also be treated with dry hydrochloric acid in ethyl acetate at room temperature. The hydrochloride of the desired diastereoisomer precipitates directly from the reaction mixture in high diastereoisomeric purity and yield.

Step 4:

The compounds of formula (I) can finally be obtained by standard decomposition methods of the salts obtained from step 3a) or by N-bocylation of the salts obtained from step 3b). Such decomposition and bocylation methods are well known to the skilled artisan. The salts of formula (IV), as obtained from step 3 of reaction sequence A can be decomposed in the presence of an inorganic base, such as for example but not limited to an alkali metal hydroxide, -hydrogencarbonate or -carbonate, preferably in the presence of sodium carbonate; followed by removal of the amine base by extraction with an organic solvent; followed by addition of a mineral acid, preferably sulfuric acid, to the remaining aqueous phase and extraction of the compounds of formula (I) into an organic solvent. Alternatively said decomposition can be achieved by direct addition of said mineral acid to the reaction mixture containing the compounds of formula (IV), followed by extraction of the compounds of formula (I) into an organic solvent.

The salts of formula (VI), as obtained from step 3 of reaction sequence B can be further N-bocylated using methods well known to the skilled artisan, preferably in the presence of an inorganic base, such as for example but not limited to an alkali metal hydroxide or -carbonate, more preferably in the presence of sodium carbonate, followed by further reaction with di-tert-butyl dicarbonate; or alternatively with di-tert-butyl dicarbonate in dichloromethane and in the presence of amine bases such as triethylamine.

Subsequently to each of the aforementioned procedures the compounds of formula (I) can finally be obtained and/or purified by crystallization from organic solvents, preferably from hexane or heptane.

The following examples are provided to aid the understanding of the present invention. It is understood that modifications can be made without departing from the spirit of the invention.

If not explicitly otherwise stated, the following abbreviations are used:
The term “min” refers to minute(s)
The term “h” refers to hour(s)
The term “rt” refers to room temperature
The term “NMR” refers to nuclear magnetic resonance
The term “GC” refers to gas chromatography
The term “TLC” refers to thin layer chromatography
The term “HPLC” refers to high performance liquid chromatography
The term “dr” refers to distereosiomer ratio
The term “er” refers to enantiomer ratio
The term “ee” refers to enantiomeric excess
The term “mp” refers to melting point

### Reaction Sequence A

### EXAMPLE 1

### Synthesis of (S)-2-(2-Benzyloxycarbonyl-propenyl)-pyrrolidine-1-carboxylic acid tert-butyl ester (2) (Synthesis with Preformed Wittig Ylide)

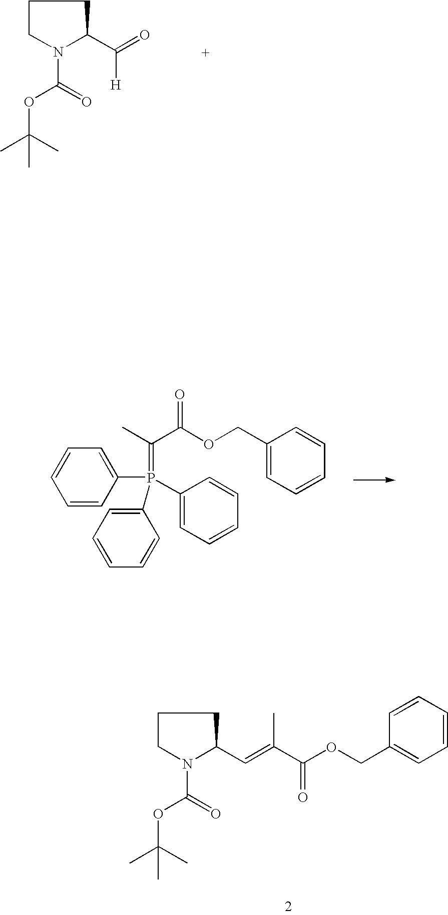

a) The Wittig ylide (benzyl 2-(triphenylphosphoranylidene)propionate) can be obtained according to the synthesis disclosed in “Y. Ito, M. Okano, R. Oda, Tetrahedron, 23, 1967, 2137”.

b) To a solution of 135.7 g benzyl 2-(triphenylphosphoranylidene)propionate (320 mmol) in 440 ml tert-butyl methyl ether was added at rt a solution of 45.5 g Boc-L-prolinal (228.4 mmol) in 62 ml tert-butyl methyl ether. The yellow solution was heated under reflux for 1.5 h upon which a white precipitate of triphenylphosphine oxide formed. From the suspension 230 ml of tert-butyl methyl ether solvent were removed by distillation using a Dean-Stark trap (a water separator used in chemical reactions). Then 360 ml heptane were added drop by drop at reflux temperature to further promote the triphenylphosphine oxide precipitation. The suspension was cooled to rt, stirred at rt overnight, then cooled to 0-5° C. and stirred at this temperature for 30 min. The suspension was filtered over a pre-cooled (0-5° C.) G3 glass filter funnel and the filter cake washed portion-wise with 250 ml pre-cooled (0-5° C.) heptane. The yellow filtrate and the wash solution were combined and evaporated (40° C./10 mbar) to provide 86.8 g of yellow oil as the crude product. GC: 4.67% Z-2, 91.55% E-2, 3.78% triphenylphosphine oxide; E/Z=95.15:4.85. Of this material 86.6 g were filtered over 434 g silica gel using ca. 3 l hexane/ethyl acetate (2:1) as the eluent to provide, after evaporation and drying in vacuo, 81.38 g (103% w/w) of the title compound (2) as light yellow oil. The material by GC analysis contained 4.59% Z-2, 90.58% E-2, and 1.12% triphenylphosphine oxide; E/Z=95.2:4.8. The material by chiral HPLC analysis contained 4.19% Z-2, 0.31% ent-E-2, and 95.50% E-2; er=99.7:0.3; E/Z=95.7:4.3.

¹H-NMR: (400 MHz, CDCl₃): 7.4-7.3 (m, 5 arom. H); 6.65 (br. d, J=7, vinyl. H of (E)-2); 5.9-5.8 (br., vinyl. H of (Z)-2); 5.3-5.1 (br. m, PhCH₂O); 4.7-4.4 (br. m, 1H); 3.6-3.35 (br. m, 2H); 2.13 (m, 1H); 2.0-1.3 [m, in total 15H, with 1.43 (br. s, tBu)].

### EXAMPLE 2

### Synthesis of (S)-2-(2-Benzyloxycarbonyl-propenyl)-pyrrolidine-1-carboxylic acid tert-butyl ester (2); (Synthesis with in situ Formation of Wittig Ylide)

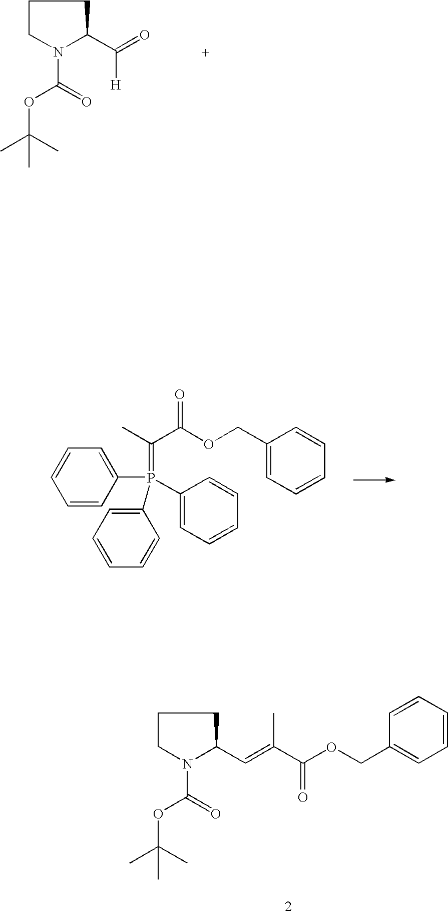

A solution of 378 g (1-benzyloxycarbonyl-ethyl)-triphenylphosphonium bromide (82.9%, 619.9 mmol) in 1.45 l dichloromethane was azeotropically distilled while keeping the volume constant by addition of 1.20 l dichloromethane. To the solution was added slowly at an internal temperature of 10-12.5° C. a solution of 71.0 g potassium tert-butoxide (98%, 620 mmol) in 640 ml tetrahydrofuran. The yellowish turbid solution was allowed to attain rt and stirred at rt for 75 min. Then, a solution of 127.4 g Boc-L-prolinal (97%, 620.3 mmol) in 640 ml tetrahydrofuran was added, whereby the reaction temperature rose to 25° C. The yellow solution was heated under reflux for 18 h upon which a white precipitate of triphenylphosphine oxide formed. The tetrahydrofuran/dichloromethane solvent mixture was exchanged for 3.6 l heptane. The suspension was then cooled to 0° C., stirred at 0° C. for 1 h, and the triphenylphosphine oxide was filtered and washed with 1 l heptane (pre-cooled at 0° C.). The combined yellow filtrate and wash solution were washed with 2×2.5 l, a total of 5 l water and evaporated (40° C./100 mbar) to provide as crude product 233.9 g of the title compound (2) as yellow oil. This material by HPLC analysis contained 89.6% E-2 and 5.4% Z-2; E/Z=94.3:5.7. The material by chiral HPLC analysis contained 5.5% Z-2, 0.0% ent-E-2, and 94.50% E-2; er=100:0; E/Z=94.5:5.5.

### EXAMPLE 3

### Synthesis of (S)-2-((1R,2S)-2-Benzyloxycarbonyl-1-methylsulfanyl-propyl)-pyrrolidine-1-carboxylic acid tert-butyl ester (3a) in mixture with (S)-2-((1R,2R)-2-Benzyloxycarbonyl-1-methylsulfanyl-propyl)-pyrrolidine-1-carboxylic acid tert-butyl ester (3b) and two further diastereoisomers of (S)-2-(2-Benzyloxycarbonyl-1-methylsulfanyl-propyl)-pyrrolidine-1-carboxylic acid tert-butyl ester of partially undetermined configuration (3c and 3d)

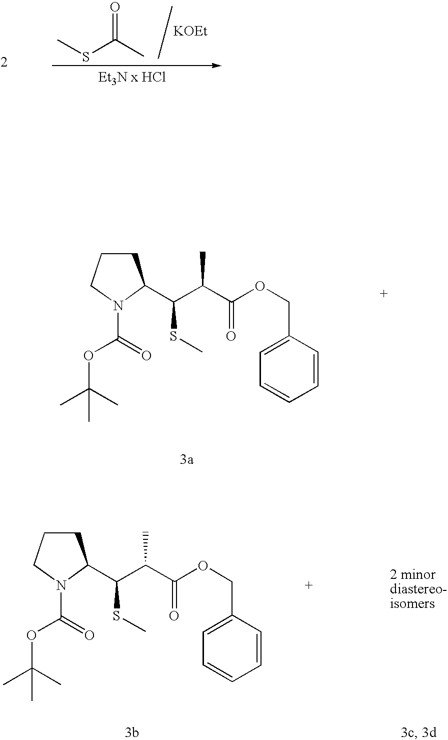

S-methyl thioacetate (64.09 g, 703 mmol) was dissolved under argon with stirring in 700 ml tetrahydrofuran. To the clear colorless solution potassium ethoxide (59.16 g, 703 mmol) was added as solid with the aid of a glass funnel and the funnel was rinsed with 100 ml tetrahydrofuran. The temperature of the yellow-orange suspension rose to 41° C. then returned to rt within 30 min. The suspension was stirred at rt for 2.75 h. After a total reaction time of 3.25 h, 48.39 g triethylamine hydrochloride (351.5 mmol) were added at once followed by dropwise addition of a solution of 80.97 g (S)-2-(2-benzyloxycarbonyl-propenyl)-pyrrolidine-1-carboxylic acid tert-butyl ester (2, from example 1) in 344 ml tetrahydrofuran. The yellow-orange suspension was stirred at rt for 5 h. For work-up 344 ml ethyl acetate and 690 ml 5M ammonium chloride solution were added at rt to the reaction mixture. The two phase system was stirred at rt for 2 min, and then transferred into a separatory funnel. The phases were separated, and the organic phase was dried over sodium sulfate, filtered and evaporated (40° C./10 mbar) to yield 93.91 g of the crude product as yellow oil. Subsequently, 93.0 g of the crude product were subjected to filtration over 465 g silica gel with ca. 2 l heptane/ethyl acetate 1:1 mixture. Evaporation and drying in vacuo afforded 91.8 g of the title compound (3) as clear yellow oil. This material by GC analysis contained 1.2% (E)-2, 84.1% 3a, 1.4% 3c, 1.5% 3d and 8.7% 3b; dr 3a/3b/3c/3d=87.8:9.1:1.5:1.6.

¹H-NMR (400 MHz, CDCl₃): 7.45-7.25 (m, 5 arom. H); 5.3-5.05 (br. m, PhCH₂O); 4.2-3.8 (br. m, 1H); 3.75-3.15 (br. m, 3H); 2.6 (br. m, 1H); 2.07 (s, SCH₃); 1.9 (m, 3H); 1.7 (m, 1H); 1.46 and 1.43 (2 s, tBu of 2 rotamers); 1.34 (d, J=6.5, CH₃).

### EXAMPLE 4

### Synthesis of (S)-2-((1R,2S)-2-Carboxy-1-methylsulfanyl-propyl)-pyrrolidine-1-carboxylic acid tert-butyl ester (1a)

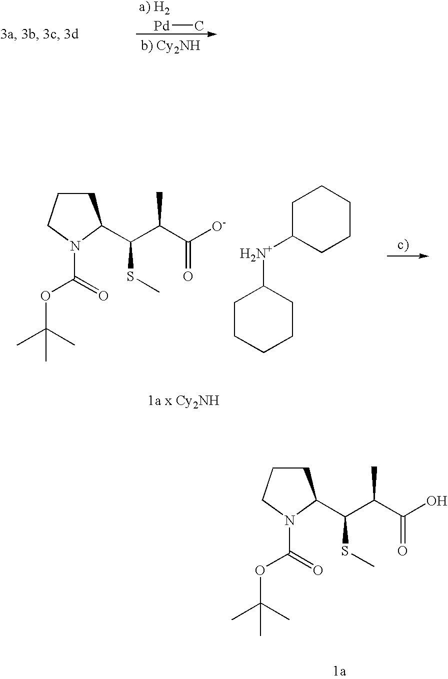

### a) Synthesis of (S)-2-((1R,2S)-2-Carboxy-1-methylsulfanyl-propyl)-pyrrolidine-1-carboxylic acid tert-butyl ester (1a) in mixture with (S)-2-((1R,2R)-2-Carboxy-1-methylsulfanyl-propyl)-pyrrolidine-1-carboxylic acid tert-butyl ester (1b) and two further diastereoisomers of (S)-2-(2-Carboxy-1-methylsulfanyl-propyl)-pyrrolidine-1-carboxylic acid tert-butyl ester of partially undetermined configuration (1c and 1d)

91.8 g (S)-2-(2-Benzyloxycarbonyl-1-methylsulfanyl-propyl)-pyrrolidine-1-carboxylic acid tert-butyl ester (3, diastereoisomer mixture, see example 3; derived from 224.5 mmol Boc-L-prolinal) were dissolved under argon in an Erlenmeyer flask in 920 ml ethanol and treated with 46.0 g Raney-Ni. The suspension was stirred at rt for 1 h, then filtered and the filter cake was thoroughly washed with 360 ml ethanol. The combined filtrate and wash solutions were divided in two parts of roughly equal volume (ca. 640 ml) which were hydrogenated separately over 13.75 g, a total of 27.5 g 20% Pd on charcoal with hydrogen at 10 bar pressure and at 30° C. for 18 h. The hydrogen uptake was 2.74 l and 2.41 l (theor. 2×2.82 l). The black suspensions of the two runs were filtered and the filter cakes were washed each with 300 ml, a total of 600 ml ethanol. The filtrates and wash solutions of both runs were combined and the solution was divided in two parts of exactly equal volumes. One part was evaporated (40° C./10 mbar/4 h) to provide, after drying in vacuo, 33.85 g of light yellow oil. The other part was concentrated to a volume of ca 150 ml, filtered to remove some traces of charcoal, and then evaporated to provide, after drying in vacuo, 33.13 g of light yellow oil. Combined yield 66.98 g of crude acid 1 (diastereoisomer mixture). This material by GC analysis contained 84.4% 1a, 1.4% 1c, 8.8% 1b and 1.65% 1d; dr 1a/1b/1c/1d=87.7:9.2:1.5:1.6. Assays of 78.1% 1a and of 7.1% 1b were determined by HPLC with internal standard.

¹H-NMR (300 MHz, CDCl₃): ca. 10 (br. s, COOH); 4.15-3.95 (br. m, 1H); 3.65-3.1 (br. m, 3H); 2.6 (br. m, 1H); 2.12 (s, SCH₃); 2.0-1.65 (m, 4H); 1.46 and 1.43 (2 s, tBu of 2 rotamers), 1.39 (d, J=6.5, CH₃).

### b) Formation of (S)-2-((1R,2S)-2-Carboxy-1-methylsulfanyl-propyl)-pyrrolidine-1-carboxylic acid tert-butyl ester; compound with dicyclohexylamine (1a×Cy₂NH)

A quantity of 33.5 g crude acid [1, diastereoisomer mixture from a), derived from 112.3 mmol Boc-L-prolinal] was dissolved in 170 ml tert-butyl methyl ether. The solution was filtered to remove some residual solid (charcoal), and then treated with 23.73 ml dicyclohexylamine (119 mmol). The solution was cooled to 0-5° C. under stirring whereby a white solid started to precipitate at ca. 8° C. The suspension was stirred at 0-5° C. for 3 h. The solid was collected by filtration over a pre-cooled glass filter funnel, washed with 100 ml pre-cooled (0-5° C.) tert-butyl methyl ether and dried (40° C./10 mbar/4 h) to furnish 38.55 g (70.8%, based on Boc-L-prolinal) of the title compound (1a×Cy₂NH) as white powder; m.p. 141-142° C.; [α]_{D}²⁰−20.56 (c 1.04, ethanol). The composition of this material as derived from GC analysis was 44.6% Cy₂NH, 54.1% 1a, 0.33% 1c, 0.69% 1b and 0.13% 1d; dr 1a/1b/1c/1d=97.9:1.25:0.6:0.25. An assay of 61% 1a (theor. 62.6%) was determined by HPLC with internal standard. Chiral HPLC analysis showed 1a to be enantiomerically pure (ent-1a not detectable).

¹H-NMR (CDCl₃, 400 MHz): 8.55 (br. s, NH₂⁺); 4.2-4.0 (br. m, 1H); 3.75-3.2 (br. m, 3H); 2.87 (m, 1H); 2.27 (m, 1H); 2.2-1.1 [m, total 39H, with 2.12 (s, SCH₃), 1.48 and 1.44, (2 s, tBu of 2 rotamers)].

### c) Isolation and Crystallization of (S)-2-(1R,2S)-2-Carboxy-1-methylsulfanyl-propyl)-pyrrolidine-1-carboxylic acid tert-butyl ester (1a)

A quantity of 38.5 g 1a×Cy₂NH (derived from 112.1 mmol Boc-L-prolinal) was treated with 160 ml tert-butyl methyl ether and 160 ml 1M sodium carbonate solution. The organic phase was separated and extracted with 160 ml 1M sodium carbonate solution. The combined aqueous phases were acidified to pH 2 by addition of 175 ml 2M sulfuric acid and the resulting mixture was extracted 3 times with 175 ml, a total of 525 ml tert-butyl methyl ether. The combined extracts were dried over ca. 90 g sodium sulfate filtered and evaporated (40° C./10 mbar/0.5 h) to provide 24.16 g of crude acid 1a as colorless viscous oil. Assays of 95.2% 1a and of 1.2% 1b were determined by HPLC with internal standard. The crude acid 1a was dissolved at rt in 120 ml hexane and the solution stirred at −20° C. for 16 h. The white precipitate was filtered over a pre-cooled (−20° C.) glass filter funnel, washed portion-wise with 60 ml hexane (pre-cooled at −20° C.) and dried (40° C./10 mbar/2 h) to furnish 19.94 g (58.5% based on Boc-L-prolinal) of the title compound (1a) as white crystals; m.p. 64.5-66° C. The material by GC analysis contained 97.9% 1a, 0.53% 1c, 0.98% 1b and 0.13% 1d; dr 1a/1b/1c/1d=98.4:1.0:0.5:0.1. Chiral HPLC analysis showed 1a to be enantiomerically pure (ent-1a not detectable).

¹H-NMR (400 MHz, CDCl₃): 4.15-3.95 (br. m, 1H); 3.65-3.15 (br. m, 3H); 2.6 (br. m, 1H); 2.12 (s, SCH₃); 1.94 (br. m, 3H); 1.75 (m, 1H); 1.47 and 1.45 (2 s, tBu of 2 rotamers), 1.39 (d, J=6.5, CH₃).

### EXAMPLE 5

### (S)-2-((1R,2S)-2-Carboxy-1-methylsulfanyl-propyl)-pyrrolidine-1-carboxylic acid tert-butyl ester; compound with (R)-1-phenyl-ethylamine (1a×(PhEt)NH₂)

Analogously to the salt formation described in example 4b) the (R)-1-phenylethyl-ammonium salt was obtained:

A quantity of 30.34 g of the crude acid 1 [diastereoisomer mixture, dr 1a/1b/1c/1d=87.7:9.2:1.5:1.6, see example 4a), derived from 101.7 mmol Boc-L-prolinal] was dissolved in 166.9 ml heptane, resulting in a slightly turbid, greenish solution. Then 12.98 g (105 mmol) (R)-(+)-1-phenyl-ethylamine were injected in one portion from a syringe resulting in a temperature increase from 25 to 35° C. The reaction mixture was stirred overnight (16 h) at rt. The precipitated crystals were separated by filtration, washed with heptane and dried in vacuo at rt, yielding 31.43 g of the crude (R)-1-phenylethylammonium salt of 1a. The crude product was purified by recrystallization from diisopropyl ether leading to 27.4 g (63% based on Boc-L-prolinal) white crystals; m.p. 99-100° C. The material contained, as derived from GC analysis, 97.7% 1a, 0.9% 1b, and 0.2 and 0.1% of the minor diastereoisomers 1c and 1d. A sample for analysis was obtained by further recrystallization, white crystals; m.p. 103-104° C.; [α]_{D}²⁰−22.4 (c 1.04, ethanol).

¹H-NMR: (300 MHz, CDCl₃): 7.5-7.2 (m, NH₃⁺ and 5 arom. H); 4.33 (q, J=6.8, PhCH(Me)); 3.97 (br. m, 1H); 3.50 (br. t, J=8, 1H); 3.24 (m, 2H); 2.45-1.15 [m, in total 23H with 2.03 (s, SCH₃), 1.58 (d, J=6.8, PHCH—CH₃), 1.34 (s, tBu), 1.20 (d, J=6.5, CH₃CH—COO⁻)].

The isolation and crystallization of 1a can be carried out analogously to the description given in example 4c).

### EXAMPLE 6

### (S)-2-((1R,2S)-2-Carboxy-1-methylsulfanyl-propyl)-pyrrolidine-1-carboxylic acid tert-butyl ester; compound with diisopropyl-amine (1a×(iPr)₂NH)

Analogously to the salt formation described in examples 4b) or 5 the diisopropyl-ammonium salt was obtained: A quantity of 1.10 g of the crude acid 1 [diastereoisomer mixture with assays of 69.9% 1a and 8.0% 1b as determined by HPLC with internal standard; cf. example 4a)] and 370 mg (3.62 mmol) of diisopropylamine were dissolved at 60° C. in 10 ml cyclohexane. The hot solution was allowed to cool to rt overnight while stirring. The precipitated white crystals were collected by filtration, washed with cyclohexane and dried at rt in vacuo to yield 700 mg (68.5%) of the diisopropyl ammonium salt of 1a as white crystals; m.p. 125-128° C.; [α]_{D}²⁰−26.9 (c 1.98, ethanol).

¹H-NMR: (300 MHz, CDCl₃): 8.42 (br. s, NH₂+); 4.1 (br. s, 1H); 3.7-3.3 (br. m, 3H); 3.24 (septet, J=6.5, 2 CHMe₂); 2.25 (m, 1H); 2.15-1.2 [m, in total 31 H, with 2.11 (s, SCH₃), 1.46 and 1.44 (2 s, tBu of 2 rotamers), 1.26 (d, J=6.5, 2 CH(CH₃)₂].

The isolation and crystallization of 1a can be carried out analogously to the description given in example 4c).

### EXAMPLE 7

### (S)-2-((1R,2S)-2-Carboxy-1-methylsulfanyl-propyl)-pyrrolidine-1-carboxylic acid tert-butyl ester; compound with (R)-1-cyclohexyl-ethylamine (1a×(CyEt)NH₂) and (S)-2-((1R,2S)-2-Carboxy-1-methylsulfanyl-propyl)-pyrrolidine-1-carboxylic acid tert-butyl ester; compound with benzyl-((R)-1-phenyl-ethyl)-amine (1a×(Bn)(PhEt)NH)

Analogously to the salt formation described in examples 4b), 5 and 6 the respective (R)-1-cyclohexyl-ethylammonium salt or benzyl-((R)-1-phenyl-ethyl)-ammonium salt can be obtained. These salts were obtained as reference examples by adding the respective amine to the pure acid (1a), which was dissolved under argon in a mixture of tert-butyl methyl ether and heptane (1:1). Stirring for 18 h at rt yields the crystalline ammonium salt which is separated by filtration, washed with heptane and dried in vacuo for about 4 h.

a) The respective (R)-1-cyclohexyl-ethylammonium salt is obtained as white crystals with a melting point of 132-133° C.; [α]_{D}²⁰−23.2 (c 1.06, ethanol).

¹H-NMR: (300 MHz, CDCl₃): 7.29 (br. s, NH₃⁺); 4.0 (br. m, 1H); 3.55 (br. t, J=8, 1H); 3.4-3.2 (m, 2H); 3.06 (qui, J=6, 1H); 2.4-1.0 [m, in total 42H with 2.09 (s, SCH₃), 1.43 (s, tBu), 1.35 (d, J=7, 1 CH₃), 1.27 (d, J=7, 1 CH₃)].

b) The respective benzyl-((R)-1-phenyl-ethyl)-ammonium salt is obtained as white crystals with a melting point of 71-73° C.; [α]_{D}²⁰−5.1 (c 1.09, ethanol).

¹H-NMR: (300 MHz, CDCl₃): 7.4-7.2 (m, 10 arom. H); 6.97 (br. s, NH₂⁺); 3.99 (q, J=5.5, 1H); 3.90 (q, J=7, 1H); 3.75 and 3.65 (AB, J=13; PhCH₂—); 3.65-3.15 (br m, 3H); 2.47 (m, 1H); 2.11 (s, SCH₃); 2.0-1.25 [m, in total 19H, with 1.46 (s, tBu), 1.36 (d, J=7, 1 CH₃)].

The isolation and crystallization of 1a can be carried out analogously to the description given in example 4c).

### Reaction Sequence B

### EXAMPLE 8

### Synthesis of (S)-2-(2-tert-Butoxycarbonyl-propenyl)-pyrrolidine-1-carboxylic acid tert-butyl ester (4) (Synthesis with preformed Wittig Ylide)

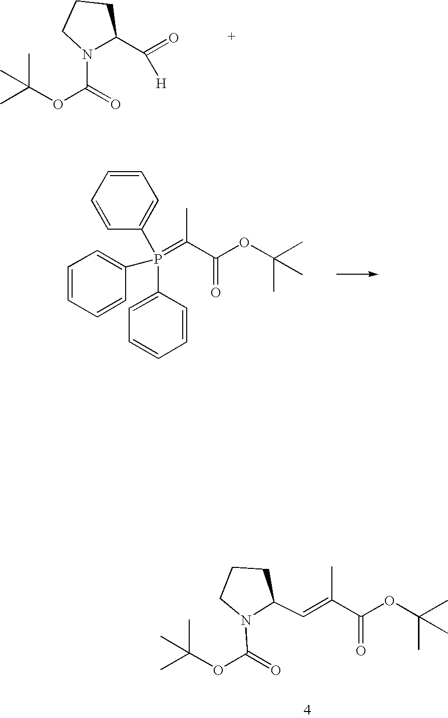

a) The Wittig Ylide ((2-triphenylphosphoranylidene)-propionic acid tert-butyl ester) can be obtained according to the synthesis described in “Y. Guindon, L. Murtagh, V. Caron, S. R. Landry, G. Jung, M. Bencheqroun, A.-M. Faucher, B. Guerin, J. Org. Chem., 66, 2001, 5427” or “P. L. Stotter, K. A. Hill, Tetrahedron Lett., 16, 1975, 1679.”

b) A quantity of 56.0 g (2-triphenylphosphoranylidene)-propionic acid tert-butyl ester (143.4 mmol) was suspended under argon and with stirring in 160 ml tert-butyl methyl ether. A solution of 21.0 g Boc-L-prolinal (105.4 mmol) in 50 ml tert-butyl methyl ether was added drop by drop. The yellowish suspension was stirred at 50° C. for 3.5 h. After complete conversion, the suspension was transferred with dichloromethane into a 1 l round bottomed flask. The solvent was removed by evaporation and final drying in vacuo (0.1 mbar/rt/15 min). The residue was taken up in 400 ml heptane, the resulting yellowish suspension stirred at rt for 30 min and the white precipitate of triphenylphosphine oxide removed by filtration over ca. 20 g decalite speed plus (diatomaceous filter-aid). The filter residue was washed 3 times with 50 ml, a total of 150 ml heptane and the combined filtrate and wash solutions were evaporated. The residue was dried (0.1 mbar/rt/2 h) to afford 34.4 g of the crude product. The material by GC analysis contained 5.5% (Z)-4, 91.7% (E)-4 and 1.8% triphenylphosphine. The crude product was dissolved in ca. 20 ml hexane/ethyl acetate (9:1 mixture) and flash-filtered over 150 g silica gel using a pressure of ca. 0.5 bar. The product was eluted with ca. 2 l hexane/ethyl acetate 9:1 mixture. Evaporation afforded as the filtered product 32.4 g (98.7% based on Boc-L-prolinal) of the title compound (4) as a light yellowish oil. The material by GC analysis contained 5.6% (Z)-4, 92.8% (E)-4 and 1.4% triphenylphosphine; E/Z=94:6. The material by chiral HPLC analysis contained 0.05% (R,E)-4 and 99.95% (S,E)-4; ee=99.9%.

¹H-NMR: (300 MHz, CDCl₃): 6.5 (br. d, J=7, vinyl. H of (E)-4); 5.75 and 5.95 (2 br. s, vinyl. H of 2 rotamers of (Z)-4); 4.65-4.35 (br. m, 1H); 3.6-3.35 (br. m, 2H); 2.15 (m, 1H); 2.0-1.3 (m, in total 24H, with 1.48 (s, tBu), 1.41 (br. s, tBu)].

### EXAMPLE 9

### Synthesis of (S)-2-((1R,2S)-2-tert-Butoxycarbonyl-1-methylsulfanyl-propyl)-pyrrolidine-1-carboxylic acid tert-butyl ester (5a) in mixture with (S)-2-((1R,2R)-2-tert-Butoxycarbonyl-1-methylsulfanyl-propyl)-pyrrolidine-1-carboxylic acid tert-butyl ester (5b) and two further diastereoisomers of (S)-2-(2-tert-Butoxycarbonyl-1-methylsulfanyl-propyl)-pyrrolidine-1-carboxylic acid tert-butyl ester of partially undetermined configuration (5c and 5d)

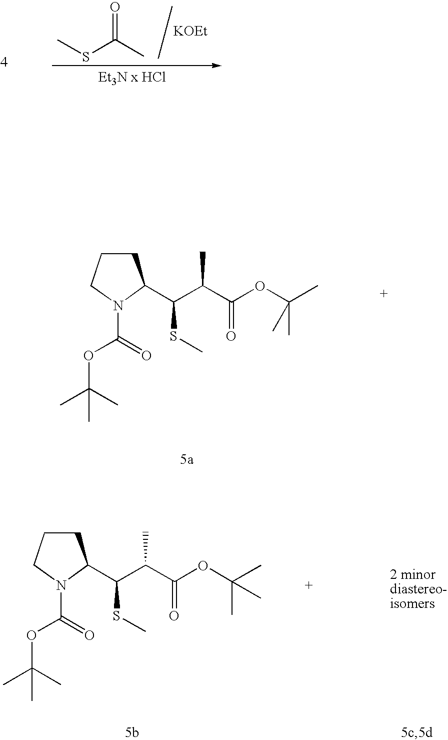

54.6 g S-methyl thioacetate (606 mmol) were dissolved under argon with stirring in 310 ml tetrahydrofuran. To the clear colorless solution 50.4 g potassium ethoxide (599 mmol) were added at once as a yellow solid with the aid of a glass funnel. The funnel was rinsed with 50 ml tetrahydrofuran. The suspension was stirred at rt for an additional 4 h. After a total reaction time of 5 h, 41.3 g triethylamine hydrochloride were added at once followed by dropwise addition of a solution of 31.1 g (S)-2-(2-tert-butoxycarbonyl-propenyl)-pyrrolidine-1-carboxylic acid tert-butyl ester (100 mmol, see example 8) in 160 ml tetrahydrofuran. The yellow suspension was stirred at rt for 22 h. After 22 h, 120 ml ethyl acetate and 350 ml 5M ammonium chloride solution were added to the reaction mixture. The two phase-system was stirred at rt for 10 min, then transferred into a separatory funnel and the phases were separated. The aqueous phase was extracted with 100 ml ethyl acetate. The combined organic phases were dried over ca. 40 g sodium sulfate, filtered and evaporated to yield 37.7 g of the crude product. The material by GC analysis contained 1.3% (Z)-4, 3.3% (E)-4, 81.8% 5a, 2.0% 5c and 9.5% of the co-eluting 5b and 5d. The crude product was dissolved in 20 ml hexane/ethyl acetate (9:1 mixture) and flash-filtered over 100 g silica gel using a pressure of ca. 0.5 bar. The product was eluted with ca. 2 l hexane/ethyl acetate 9:1 mixture. Evaporation and drying (0.1 mbar/rt/2 h) afforded as the filtered product 35.5 g of the title product 5 (99% based on Boc-L-prolinal) as a clear yellow oil. This material consisted by GC analysis of 1.3% (Z)-4, 2.6% (E)-4, 82.5% 5a, 2.2% 5c, 7.5% 5b and 2.2% 5d; dr 5a/5b/5c/5d=87.4. 8.0:2.3:2.3.

¹H-NMR: (300 MHz, CDCl₃): 4.2-3.1 (br. m, 4H); 2.45 (m, 1H); 2.3-1.15 (m, in total 28H, with 2.11 (s, SCH₃), 1.48 and 1.46 (2 s, tBu of 2 rotamers), 1.29 (br. d, J=6.5, CH₃)].

### EXAMPLE 10

### Synthesis of (S)-2-((1R,2S)-2-Carboxy-1-methylsulfanyl-propyl)-pyrrolidinium chloride (6a×HCl)

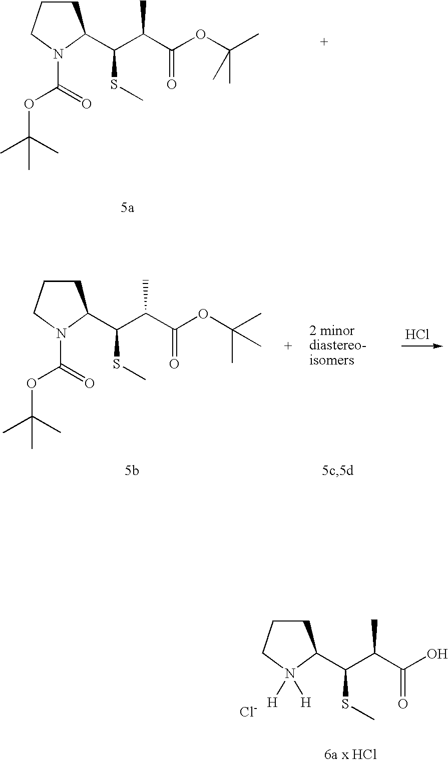

A quantity of 33.5 g (S)-2-(2-tert-butoxycarbonyl-1-methylsulfanyl-propyl)-pyrrolidine-1-carboxylic acid tert-butyl ester (5, 93.3 mmol, from example 9; diastereoisomer mixture, 5a/5b/5c/5d=87.4:8.0:2.3:2.3) was dissolved under argon with stirring in 185 ml of a 4.44M solution of dry hydrochloric acid in ethyl acetate (821 mmol). The solution was stirred at rt for 30 min, then seed crystals were added whereby crystallization started. The suspension was stirred at rt for 2 h and at 0° C. for 2 h. The precipitate was isolated by filtration, washed two times with 10 ml, a total of 20 ml cold ethyl acetate (0° C.) and dried in vacuo (0.1 mbar) at rt for about 18 h to afford 15.5 g (69% based on 5) of the title compound (1a×HCl) as white crystals; m.p. 169-170° C.

¹H-NMR (300 MHz, d₆-DMSO): 12.5 (br. s, COOH); 9.9 and 8.9 (2 br. s, NH₂+); 3.57 (q, J=6.7, 1H); 3.34 (dxd, J=9 and 4.5, 1H); 3.21 (m, 2H); 2.86 (m, 1H); 2.25 (m, 1H); 2.19; (s, SCH₃); 2.0-1.65 (m, 3H); 1.15 (d, J=6.9, CH₃).

### EXAMPLE 11

### Synthesis of (S)-2-(1R,2S)-2-Carboxy-1-methylsulfanyl-propyl)-pyrrolidine-1-carboxylic acid tert-butyl ester (1a)

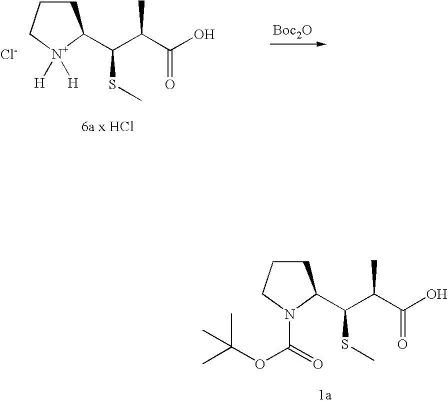

A quantity of 15.2 g (S)-2-((1R,2S)-2-carboxy-1-methylsulfanyl-propyl)-pyrrolidinium chloride (6a×HCl, 63.4 mmol, from example 10) was suspended under argon with stirring in 280 ml dioxane. A solution of 9.4 g sodium carbonate (89 mmol) in 205 ml deionized water was added. Upon addition of approximately half of the volume, a clear solution formed which after completion of the addition turned into a milky solution. Then 17.3 g di-tert-butyl dicarbonate (79.3 mmol) were added and the slightly turbid solution was stirred at rt for 5.5 h. For work-up 100 ml tert-butyl methyl ether/heptane 1:1 mixture were added and the two phases were separated. The organic phase was evaporated to afford 4.4 g of a colorless oil containing product by TLC and HPLC. The aqueous phase was overlaid with 150 ml tert-butyl methyl ether and acidified under stirring with 57.5 ml 2N hydrochloric acid to pH 2. After phase separation, the water phase was extracted 3 times with 40 ml, a total of 120 ml tert-butyl methyl ether. The combined organic phases were washed 2 times with 40 ml, a total of 80 ml saturated sodium chloride solution, dried over ca. 40 g sodium sulfate, filtered and evaporated. The residue was taken up in little dichloromethane and combined with the 4.4 g material obtained above. The solution was evaporated and the residue dried in vacuo (0.1 mbar) at a temperature of 70° C. for 2 h to yield 21.5 g of crude product as thick colorless oil. The material by GC analysis consisted of 96.0% 1a, 0.35% 1c, 0.43% 1b and 0.17% 1d; dr 1a/1b/1c/1d=99.0:0.5:0.4:0.1. HPLC analysis with internal standard indicated an assay of 85.0 w % 1a. For crystallization the crude material was dissolved in 60 ml heptane at 70°. The clear solution was stirred and allowed to cool to rt whereby crystallization started after ca. 20 min. The suspension was stirred at 0° C. for 3 h, and the resulting thick suspension placed in the refrigerator at 4° C. for 24 h and finally in the freezer at −18° C. for 72 h. The precipitate was isolated by filtration, washed 2 times with 10 ml, a total of 20 ml cold heptane and dried in vacuo (0.1 mbar) at rt for 2 h to afford as the 1^{st} crop product 15.6 g (81%) of 1a as white crystals; m.p. 71-72° C. The material by GC analysis consisted of 98.9% 1a, 0.25% 1c, 0.04% 1b and 0.00% 1d; dr 1a/1b/1c/1d=99.7:0.05:0.25:0.0.

¹H-NMR (300 MHz, CDCl₃): ca. 10 (br. s, COOH); 4.15-3.95 (br. m, 1H); 3.65-3.1 (br. m, 3H); 2.6 (br. m, 1H); 2.12 (s, SCH₃); 2.0-1.65 (br. m, 4H); 1.46 (br. s, tBu), 1.39 (br. d, J=6.5, CH₃). Microanalysis calc. for C₁₄H₂₅NO₄S (303.42): C, 55.42; H, 8.30; N, 4.62, S, 10.57; found: C, 55.34/55.25, H, 7.88/7.88, N, 4.64/4.64, S, 10.56/10.59.

The residue from the mother liquor (4.3 g, colorless oil) was dissolved in a round bottom flask in 9 ml heptane at 70°, and the solution was allowed to cool to rt. Seed crystals from the 1^{st} crop were added and the flask was placed in a freezer at −18° for 48 h. Filtration and drying as described above afforded as the 2^{nd} crop product 1.04 g (5.4%) of 1a as white crystals; m.p. 70-71° C. The material by GC analysis consisted of 98.2% 1a, 0.60% 1c, 0.13% 1b and 0.10% 1d; dr 1a/1b/1c/1d=99.2:0.1:0.6:0.1.

¹H-NMR (300 MHz, CDCl₃): identical with ¹H-NMR of 1^{st} crop material. Combined yield: 16.64 g 1a (86.5%)

Unless stated to the contrary, all compounds in the examples were prepared and characterized as described. All ranges recited herein encompass all combinations and subcombinations included within that range limit. All patents and publications cited herein are hereby incorporated by reference in their entirety.

1. A process for the manufacture of the compounds of formula (I): 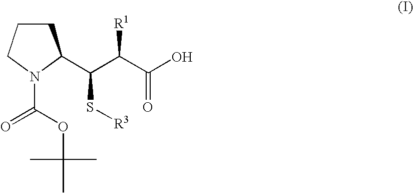
comprising:
(a) reacting a compound of formula (II): 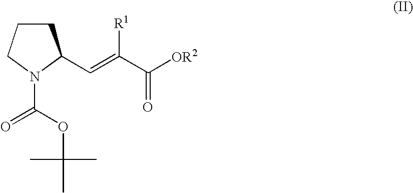
with a compound of formula (III):
KS—R³ (III),
in the presence of triethylammonium chloride in a suitable solvent, wherein said compound of formula (III) can optionally be generated in situ by reacting a compound of formula (III-A) in the presence of potassium bases wherein formula (III-A) is: 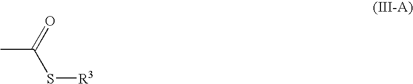
(b) chemically cleaving R² in the —COOR² ester group of the reaction product of step (a);
(c) adding an amine of the formula NHR⁴R⁵ to the resulting carboxylic acid of step (b) to form an ammonium salt of formula (IV): 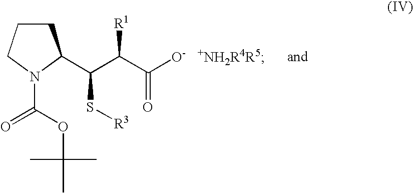
(d) decomposing said salt of formula (IV) in step (c) to form a compound of formula I;
wherein:
R¹, R³ and R⁶ independently from each other represent alkyl;
R² is benzyl or substituted benzyl; and
R⁴ and R⁵ are independently selected from cycloalkyl or alkyl, wherein said alkyl can be unsubstituted or substituted one, two or three times with hydroxy, alkoxy, amino, mono-alkylamino, di-alkylamino, acetoxy, alkylcarbonyloxy, carbamoyloxy, alkoxycarbonyl, carbamoyl, alkylcarbamoyloxy, halogen, cycloalkyl or phenyl.

2. A process for the manufacture of the compounds of formula (I): 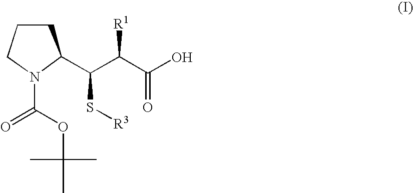
comprising:
(a) reacting a compound of formula (V) 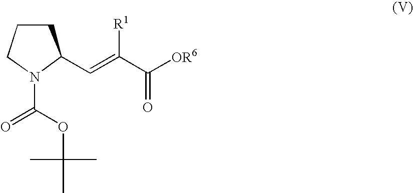
with a compound of formula (III):
KS—R³ (III),
wherein said compound of formula (III) can optionally be generated in situ by reacting a compound of formula (III-A) in the presence of potassium bases wherein formula (III-A) is: 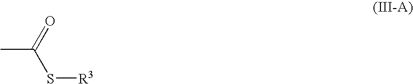
(b) adding hydrochloric acid to the reaction product of step (a) to form a compound of formula (VI) 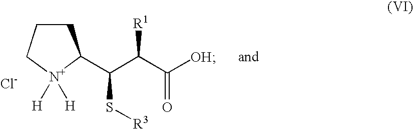
(c) reacting the reaction product of step (b) with a tert-butoxycarbonyl-delivering reagent to form a compound of formula I; wherein:
R¹, R³ and R⁶ independently from each other represent alkyl;
R² is benzyl or substituted benzyl; and
R⁴ and R⁵ are independently selected from cycloalkyl or alkyl, wherein said alkyl can be unsubstituted or substituted one, two or three times with hydroxy, alkoxy, amino, mono-alkylamino; di-alkylamino, acetoxy, alkylcarbonyloxy, carbamoyloxy, alkoxycarbonyl, carbamoyl, alkylcarbamoyloxy, halogen, cycloalkyl or phenyl.

3. The process according to claim 1, comprising:
(a) reacting the compounds of formula (II-A): 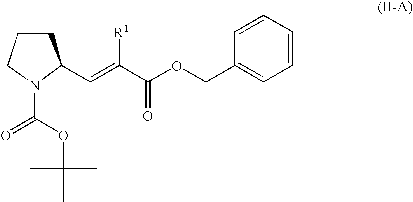
with: (1) a compound of formula (III) in the presence of triethylammonium chloride in tetrahydrofuran; or (2) a compound of formula (III-A) together with a potassium base in the presence of triethylammonium chloride in tetrahydrofuran;
(b) chemically cleaving the benzyl-ester group from the reaction product of step (a);
(c) adding an amine of the formula NHR⁴R⁵ to the resulting carboxylic acid of step (b);
(d) adding a base to the reaction product of step (c); and
(e) adding one or more mineral acids the reaction product of step (d).

4. The process according to claim 1, wherein the amines of formula NHR⁴R⁵ are selected from the group consisting of: dicyclohexylamine, diisopropylamine, (R)-α-phenylethylamine, benzyl-(R)-α-phenylethylamine and (R)-α-cyclohexylethylamine.

5. The process according to claim 2, comprising:
(a) reacting the compound of formula (2): 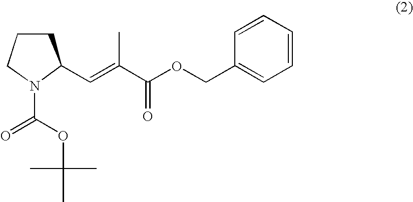
with S-methyl thioacetate together with potassium ethoxide, in the presence of triethylammonium chloride in tetrahydrofuran;
(b) chemically cleaving the benzyl-ester group from the reaction product of step (a);
(c) adding dicyclohexylamine to the resulting carboxylic acid of step (b);
(d) adding sodium carbonate to the reaction product of step (c); and
(e) adding sulfuric acid to the reaction product of step (d);
to obtain the compound of formula (1a):

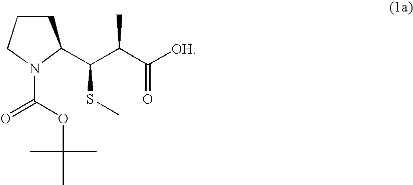

6. The process according to claim 2, comprising:
(a) reacting a compound of formula (V-A): 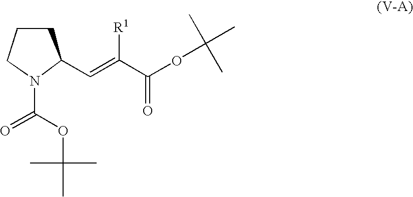
with: (1) a compound of formula (III) in the presence of triethylammonium chloride in tetrahydrofuran; or (2) a compound of formula (III-A) together with a potassium base in the presence of triethylammonium chloride in tetrahydrofuran;
(b) reacting the reaction product of step (a) with dry hydrochloric acid in ethyl acetate,
(c) adding sodium carbonate to the reaction product of step (b); and
(d) reacting the reaction product of step (c) with di-tert-butyl dicarbonate.

7. The process according to claim 6, comprising:
(a) reacting a compound of formula (4): 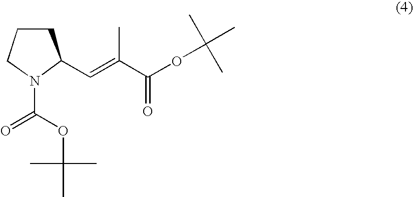
with S-methyl thioacetate together with potassium ethoxide, in the presence of triethylammonium chloride in tetrahydrofuran;
(b) reacting the reaction product of step (a) with dry hydrochloric acid in ethyl acetate;
(c) adding sodium carbonate to the reaction product of step (b); and
(d) reacting the reaction product of step (c) with di-tert-butyl dicarbonate to obtain the compound of formula (1a):

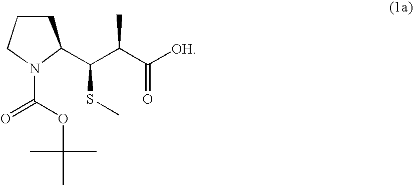

8. A process for the manufacture of the compounds of formula (A): 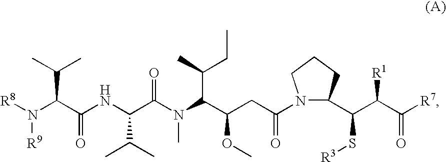
comprising:
(a) reacting a compound of formula (I) as defined in claim 1 with an alcohol or an amine, and then chemically cleaving the tert-butoxycarbonyl group at the pyrrolidine N-atom, to obtain the compounds of formula (B): 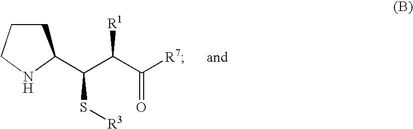
(b) further reacting the compounds of formula (B) with the compounds of formula (C): 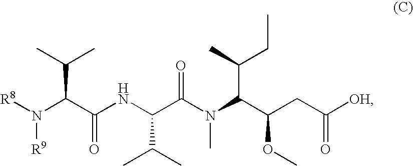
to obtain the compounds of formula (A); and wherein:
R¹ and R³ independently from each other represent alkyl;
R⁸ and R⁹ independently from each other represent alkyl; and
R⁷ is phenyl(C₁-C₄)alkyl-, or phenyldi(C₁-C₄)alkylamino or phenyl(C₁-C₄)alkyloxy, wherein the phenyl group optionally may be substituted with one, two or three substituents selected from the group consisting of halogen, alkoxycarbonyl, sulfamoyl, alkylcarbonyloxy, carbamoyloxy, cyano, mono-alkylamino, di-alkylamino, alkyl, alkoxy, phenyl, phenoxy, trifluoromethyl, trifluoromethoxy, alkylthio, hydroxy, alkylcarbonylamino, 1,3-dioxolyl, 1,4-dioxolyl, amino and benzyl.

9. A process for the manufacture of the compounds of formula (A-1): 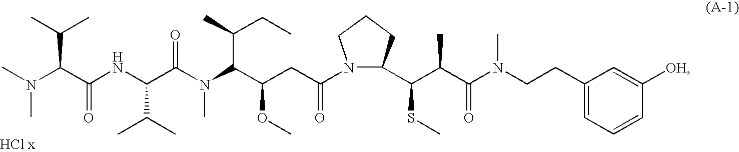
comprising:
(a) reacting the compound of formula (1a): 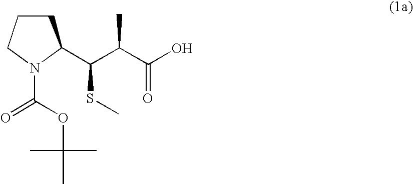
with 3-(2-methylamino-ethyl)-phenol;
(b) chemically cleaving the tert-butoxycarbonyl group at the pyrrolidine N-atom, to obtain the compound of formula (B-1): 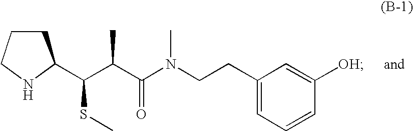
(c) reacting the compound of formula (B-1) with the compound of formula (C-1): 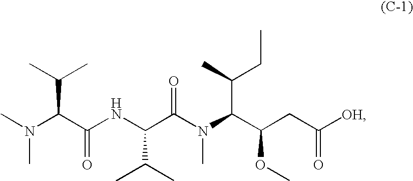
to obtain the compound of formula (A-1).

10. A compound of formula (A) as defined in claim 8 or a pharmaceutically acceptable salt thereof made by a process according to claim 8.

11. A compound of formula (A-1) as defined in claim 9 made by a process according to claim 9.

12. The compounds of formula (IV): 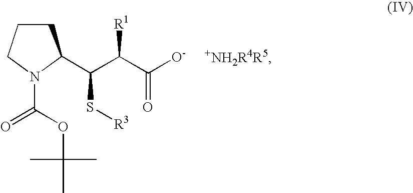
wherein R¹ and R³ independently from each other represent alkyl; and
R⁴ and R⁵ are independently selected from the group consisting of: (a) cycloalkyl and (b) alkyl;
wherein said alkyl can be unsubstituted, or substituted one, two or three times with hydroxy, alkoxy, amino, mono-alkylamino, di-alkylamino, acetoxy, alkylcarbonyloxy, carbamoyloxy, alkoxycarbonyl, carbamoyl, alkylcarbamoyloxy, halogen, cycloalkyl or phenyl.

13. The compounds according to claim 12, wherein R¹ and R³ are methyl; and the group ⁺NH₂R⁴R⁵ represents a cation selected from the group consisting of: dicyclohexylammonium, diisopropylammonium, (R)-α-phenylethylammonium, benzyl-(R)-α-phenylethylammonium, and (R)-α-cyclohexylethylammonium.

14. The compounds of formula (VI): 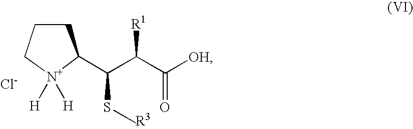
wherein R¹ and R³ independently from each other represent alkyl.

15. The compound according to claim 14, wherein R¹ and R³ are methyl.

16. The compound (S)-2-((1R,2S)-2-Carboxy-1-methylsulfanyl-propyl)-pyrrolidine-1-carboxylic acid tert-butyl ester.

17. A compound of formula (I) made by a process according to claim 1.

18. A compound of formula (I) made by a process according to claim 3.

19. A compound of formula (I) made by a process according to claim 4.

20. A compound of formula (I) made by a process according to claim 2.

21. A compound of formula (I) made by a process according to claim 5.

22. A compound of formula (I) made by a process according to claim 6.

23. A compound of formula (I) made by a process according to claim 7.

24. A pharmaceutical composition comprising a therapeutically effective amount of a compound of claim 10 and a pharmaceutically acceptable carrier.

25. A pharmaceutical composition comprising a therapeutically effective amount of a compound of claim 11 and a pharmaceutically acceptable carrier.
